(19) United States
(12) Patent Application Publication
(10) Pub. No.: US 2012/0252102 A1
(43) Pub. Date: Oct. 4, 2012

(54) BIOREMEDIATION SYSTEMS, COMPOSITIONS, AND METHODS

(75) Inventor: Troy John Fowler, Beaverton, OR (US)

(73) Assignee: Bioremediation Specialists, LLC, Beaverton, OR (US)

(21) Appl. No.: 13/436,532

(22) Filed: Mar. 30, 2012

Related U.S. Application Data
(60) Provisional application No. 61/470,284, filed on Mar. 31, 2011.

(51) Int. Cl. C02F 3/00; C12S 99/00; C12N 1/18

(52) U.S. Cl. 435/255.2; 210/170.07; 435/262

(57) ABSTRACT

Systems, compositions, and methods for the anaerobic oxidative bioremediation of a contaminant contained within a treatment zone associated with a contaminated region. These systems, compositions, and methods may include a bioremediation formulation that includes a high-mobility oxidant, a low-mobility oxidant, and a nutrient source. The high-mobility oxidant may include a nitrate salt. The low-mobility oxidant may include a sulfate salt. The nutrient source may include brewer's yeast and/or a complex sugar. The bioremediation formulation also may include one or more additional components, including a phosphate salt, a surfactant, a solvent, a chemical oxidant, and/or a bio-augmentation species. Methods of supplying the bioremediation formulation to the treatment zone may include identifying the contaminated region, estimating a mass of contaminant within the contaminated region, determining a bioremediation formulation composition, supplying the bioremediation formulation to the treatment zone, supplying supplemental material(s) to the treatment zone, and/or modifying the environment within the treatment zone.

### FIELD

The present disclosure is directed to systems, compositions, and methods for bioremediation of a contaminated region.

### BACKGROUND

The natural environment, including water and/or soil, may become contaminated with contaminants, illustrative, non-exclusive examples of which include hydrocarbons, organic solvents, pesticides, herbicides, metals, and partially halogenated solvents. These contaminants may be detrimental to a natural ecosystem that may interact with a contaminated region, or contaminated material, and/or may pose health hazards for humans, wildlife, the environment, ecosystems, and/or animals. Microorganisms, such as bacteria and fungi, may consume a portion of these contaminants as part of their natural respiratory processes. This consumption may decompose or degrade the contaminants into less harmful and/or benign respiration products, decreasing contaminant concentration within, or cleaning, the contaminated region, and may take place using aerobic and/or anaerobic reaction pathways. In aerobic respiration, molecular oxygen serves as the ultimate electron acceptor, or oxidant, for the respiratory process, while in anaerobic respiration, another chemical compound serves as the ultimate electron acceptor, or oxidant, for the respiratory process.

Bioremediation is the targeted and deliberate use of these biological, or respiratory, processes to degrade, consume, break down, transform, metabolize, and/or remove contaminants from a treatment zone that is associated with a contaminated region and may be performed both in situ and/or ex situ. In situ bioremediation includes treating the contaminated material without removal from its current, existing, or natural location, while ex situ bioremediation includes removal of the contaminated material from its current, existing, or natural location for treatment at a different site. Bioremediation processes that include the introduction of reactants for the respiration process, such as oxidants and/or nutrients, into the treatment zone to enhance, assist, augment, stimulate, and/or promote the growth of native microorganisms that are already present within the contaminated region are termed bio-stimulation processes, while bioremediation processes that include the introduction of non-native microorganisms into the treatment zone, with or without the introduction of oxidants and/or nutrients, are termed bio-augmentation processes.

For bioremediation to occur, a contaminated region must include a microbial population that is adapted to metabolize a contaminant, as well as an energy source, a carbon source, an electron acceptor (or oxidant), nutrients, and suitable environmental conditions. The microbial population may include native microbes and/or may include specialized microbes that may be added to the treatment zone during a bio-augmentation process. The contaminant is typically utilized by the microbial population as both the energy source and the carbon source, providing the mechanism by which the bioremediation processes may decrease a contaminant concentration within the treatment zone.

Once a suitable microbial population is present within the treatment zone, bio-stimulation processes may be utilized to increase a rate of contaminant consumption by the microbial population, such as by providing a source of oxidants and/or nutrients and/or by providing an environment that is more suitable for microbial growth. Illustrative, non-exclusive examples of environmental conditions that may impact microbial growth may include the temperature, pH, salinity, pressure, contaminant concentration, and/or an inhibitor concentration within the treatment zone.

### SUMMARY

The present disclosure is directed to bioremediation compositions, or formulations, and to systems and methods of using those bioremediation formulations. These bioremediation formulations are configured to provide at least a portion of the oxidants and nutrients that are consumed by a native microbe population during anaerobic respiration to promote the anaerobic oxidative bioremediation of a contaminant contained within a treatment zone associated with a contaminated region. The bioremediation formulations include one or more high-mobility oxidants, one or more low-mobility oxidants, and a nutrient material. The high-mobility oxidant may include any suitable chemical compound that may be highly mobile within the treatment zone. Conversely, the low-mobility oxidant may include any suitable chemical compound that may be less mobile within the treatment zone when compared to the high-mobility oxidant. In some embodiments, the high-mobility oxidant includes at least one nitrate salt. In some embodiments, the low-mobility oxidant includes at least one sulfate salt. In some embodiments, the nutrient material includes at least one complex sugar. In some embodiments, the nutrient material includes brewer's yeast. In some embodiments, the bioremediation formulations also may include a mid-mobility oxidant, which may be more mobile than the low-mobility oxidant but less mobile than the high-mobility oxidant. In some embodiments, the mid-mobility oxidant may include a sulfate salt. In some embodiments, the bioremediation formulations also may include at least a first additional component. In some embodiments, the at least a first additional component includes at least one of an ionic surfactant, a non-ionic surfactant, a co-solvent, a chemical oxidant, and/or a bio-augmentation species. In some embodiments, the bioremediation formulation also may include at least a first phosphate salt. The bioremediation formulations may be supplied to the treatment zone in any suitable state, including as a dry powder and/or as an aqueous solution or suspension. In some embodiments, a mass of bioremediation formulation supplied to the treatment zone may be calculated based at least in part on a mass of contaminant contained within, or estimated to be contained within, the contaminated region. In some embodiments, the contaminated region may include at least one of a soil sample and an aqueous environment.

### BRIEF DESCRIPTION OF THE DRAWINGS

FIG. 1 is a schematic illustration of an illustrative, non-exclusive example of a bioremediation formulation according to the present disclosure.

FIG. 2 is a schematic illustration of an illustrative, non-exclusive example of a treatment zone associated with a contaminated region that may be utilized with the systems and methods according to the present disclosure.

FIG. 3 is a flowchart schematically illustrating illustrative, non-exclusive examples of bioremediation methods according to the present disclosure.

### DETAILED DESCRIPTION

The systems, compositions, and methods disclosed herein include a bioremediation formulation that is configured or adapted to provide at least a portion of the oxidants and nutrients that are utilized by a microbial population to support anaerobic respiration and are illustrated in the included Figures. Unless indicated otherwise, elements shown in dashed lines, or indicated with dashed lead lines, are considered to be optional features, structures, and/or steps. Elements shown in solid lines, or indicated with solid lead lines, are typically included in the systems, compositions, and methods disclosed herein; however, elements shown in dashed lines and/or those shown in solid lines may be omitted from a particular embodiment without departing from the scope of the present disclosure. In addition, the individual features, structures, and/or steps disclosed herein may be organized in any suitable fashion without departing from the scope of the present disclosure.

The systems, compositions, and methods disclosed herein promote the anaerobic oxidative bioremediation of a contaminant that is contained within a contaminated region by stimulating the normal life function of a microbial population that is associated with, present within, and/or naturally occurring within the contaminated region. The stimulating includes supplying at least a portion of the oxidants and nutrients that are used by the microbial population as part of its natural respiratory process. The disclosed systems, compositions, and methods encourage the consumption of the contaminant as a carbon and/or energy source, which also may be referred to herein as a food source, for the microbial population, thereby decreasing the concentration of the contaminant within the contaminated region.

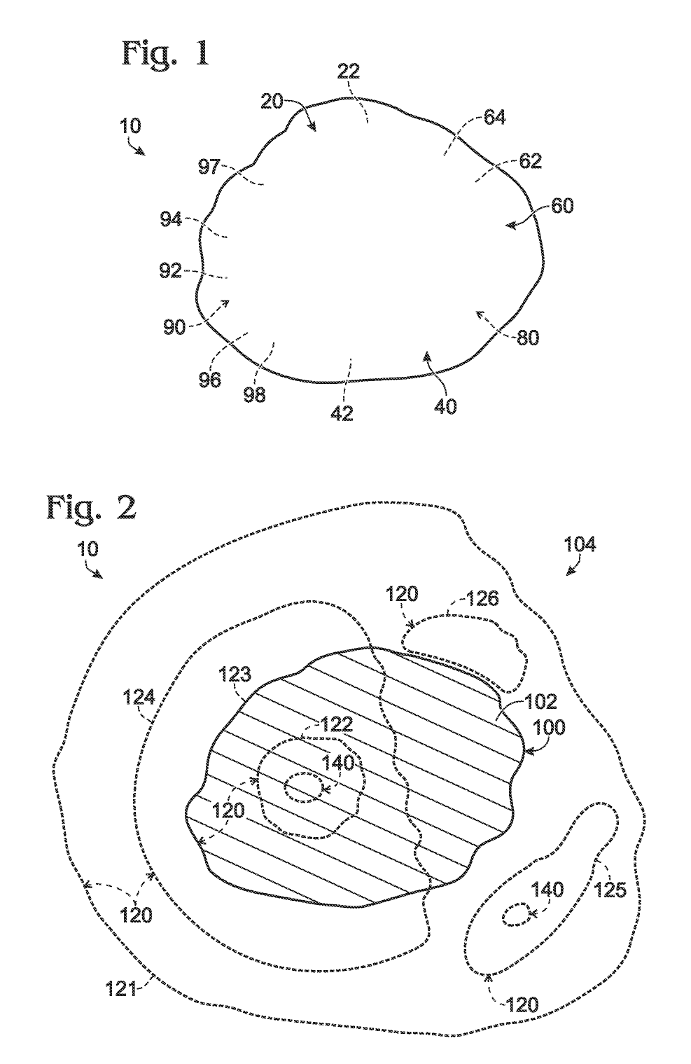

FIG. 1 provides a schematic representation of an illustrative, non-exclusive example of bioremediation formulations 10 according to the present disclosure. The bioremediation formulations include at least a high-mobility oxidant 20, a low-mobility oxidant 40, and a nutrient material 60. In addition, and as shown in dashed lines in FIG. 1, the bioremediation formulations optionally may include a mid-mobility oxidant 80 and/or at least a first additional component 90. As discussed in more detail herein, each of the individual components included within bioremediation formulations 10 may be present in any suitable amount, proportion, or percentage, including the illustrative, non-exclusive examples presented herein.

Bioremediation formulations 10 may include both active and inactive components. As used herein, active components may refer to components of the bioremediation formulation that are actively utilized during microbial respiration and/or components of the bioremediation formulation that actively participate in the delivery of the bioremediation formulation to the microorganisms, contaminant, treatment zone, and/or contaminated region. In contrast, inactive components may refer to components of the bioremediation formulation that are not actively utilized during microbial respiration and/or components of the bioremediation formulation that do not actively participate in the delivery of the bioremediation formulation to the microorganisms, contaminant, treatment zone, and/or contaminated region.

The amount, proportion, or percentage of a particular component of the bioremediation formulation may be expressed as a weight percentage (wt %) of that component. It is within the scope of the present disclosure that, when the bioremediation formulation includes inactive components, the weight percentage may refer to a weight percentage of the active components contained within the bioremediation formulation and may not include the weight of the inactive components contained therein. Furthermore, when the bioremediation formulation forms a liquid solution, an illustrative, non-exclusive example of which is an aqueous solution, it is within the scope of the present disclosure that the weight percentage may refer to the weight percentage of the solute contained within the liquid solution and/or a weight percentage of the dry components that are contained within the liquid solution.

As used herein, the terms microbe and microorganism may be used interchangeably and may refer to any suitable microscopic life form that may be present within the treatment zone and/or the contaminated region and may be adapted to consume, degrade, or otherwise decompose contaminants contained therein. Illustrative, non-exclusive examples of microorganisms according to the present disclosure include any suitable bacteria, such as pseudomonas and/or E. coli, fungi, denitrifier, sulfate reducer, anaerobic species, facultative anaerobic species, and/or facultative aerobic species. Illustrative, non-exclusive examples of contaminants according to the present disclosure include hydrocarbons, petroleum hydrocarbons, metals, partially halogenated solvents, benzene, ethyl benzene, toluene, xylene, gasoline, diesel, oil, and/or vinyl chloride.

As discussed in more detail herein, the systems, compositions, and methods disclosed herein may include multiple oxidants that include multiple oxidant mobilities within the treatment zone, as well as multiple oxidant energy states. This variation in oxidant mobility and oxidant energy state may provide for stimulation and/or growth of a wide variety of microorganisms over both long and short time frames and covering both long and short length scales within the treatment zone, providing a more effective overall bioremediation treatment. As an illustrative, non-exclusive example, the systems, compositions, and methods disclosed herein may stimulate the simultaneous growth of a plurality of microorganisms, including denitrifying, iron-related, and/or sulfate reducing bacteria to simultaneously consume a plurality of contaminants, including metals, benzene, ethyl benzene, toluene, and/or xylene.

As used herein, anaerobic respiration refers to respiratory processes that occur without the consumption, or without substantial consumption, of molecular oxygen. Instead, these anaerobic respiratory processes include the use of another suitable electron acceptor, or oxidant, to accept the electrons that are removed from the food source during the respiratory process. Illustrative, non-exclusive examples of suitable oxidants according to the present disclosure include nitrate ions, ionized metals, such as manganese (IV) and/or iron (III), sulfate ions, and/or carbon dioxide.

In general, anaerobic respiratory processes are less efficient, or slower, than aerobic respiratory processes, which utilize molecular oxygen as an electron acceptor, since a greater amount of energy is available to the microbial population when oxygen is utilized as an electron acceptor. However, certain microbes may only be capable of performing anaerobic respiratory processes. Also, certain treatment zones may contain little or no naturally occurring molecular oxygen and supplying molecular oxygen to the treatment zone may not be practical. In addition, and when the treatment zone includes an aqueous environment, a solubility of molecular oxygen within the aqueous environment may be orders of magnitude less than a solubility of another suitable oxidant, such as a salt, within the aqueous environment. Under these conditions, anaerobic respiratory processes may proceed more rapidly than aerobic respiratory processes due to the greater availability of reactants for the anaerobic respiratory processes within the aqueous environment and may therefore provide a more efficient overall bioremediation process.

Additionally or alternatively, and although anaerobic respiratory process may be less efficient, or slower, that aerobic respiratory processes, the systems, compositions, and methods disclosed herein may provide for a rapid increase in a biomass of microbes within the treatment zone to a level that is significantly higher than a biomass that may be sustainable and/or supported by aerobic respiratory processes. This increase in microbe biomass may promote metabolism of contaminants that are contained within the treatment zone at a rate that is greater than a rate that may be attained using aerobic respiratory processes.

As used herein, the phrase anaerobic oxidative bioremediation refers to a bioremediation process that encourages anaerobic microbial respiration as a mechanism to promote the oxidation of, or removal of electrons from, the contaminant. As an illustrative, non-exclusive example, this may be accomplished by providing an abundance, or excess, of oxidants for the microbes to utilize during their respiratory processes.

As used herein, a high-mobility oxidant is a relative term that refers to an oxidant that has a higher mobility, or diffusion constant, within the treatment zone when compared to a mid-mobility oxidant or a low-mobility oxidant. Similarly, a mid-mobility oxidant is a relative term that refers to an oxidant that has a higher mobility, or diffusion constant, within the treatment zone when compared to the low-mobility oxidant but a lower mobility, or diffusion constant, within the treatment zone when compared to the high-mobility oxidant. The differences in oxidant mobilities may be caused by a variety of factors, including oxidant molecule diffusion constants, oxidant solubility, and/or oxidant affinity for one or more materials present within the treatment zone.

As an illustrative, non-exclusive example, high-mobility oxidants, such as nitrate salts, may be more soluble in water that may be present within the treatment zone when compared to mid or low-mobility oxidants, such as sulfate salts. As another illustrative, non-exclusive example, and when the treatment zone includes soil, stone, and/or other geological structures, high-mobility oxidants may have a lower affinity for and/or attraction to the geological structures when compared low-mobility oxidants. As yet another illustrative, non-exclusive example, mid and low-mobility oxidants, such as sulfate salts, may form low-solubility complexes with chemical species that are already present within the treatment zone, while high-mobility oxidants, such as nitrate salts, may be less likely to form the low-solubility complexes.

As discussed in more detail herein, the systems, compositions, and methods disclosed herein may include a mixture of oxidants that may include a range of oxidant mobilities within the treatment zone. This may include low-mobility oxidants, which may remain in, or substantially in, the portion of the treatment zone to which they are applied, as well as mid- and/or high-mobility oxidants, which may diffuse more rapidly throughout the treatment zone. The use of a mixture of oxidants that include a range of oxidant mobilities may provide localized, concentrated, and/or short timeframe bioremediation within an application site as well as a more dispersed bioremediation over longer distances and/or times.

High-mobility oxidants include any suitable composition, other than molecular oxygen, that is adapted to function as an oxidant, or electron acceptor, during the anaerobic microbial respiration process and that is highly mobile within the treatment zone when compared to low- and/or mid-mobility oxidants. The high-mobility oxidant may comprise any suitable proportion of a bioremediation formulation 10, illustrative, non-exclusive examples of which include high-mobility oxidants that comprise 0-50 wt % of the bioremediation formulation, optionally comprising 0-40 wt %, 1-50 wt %, 1-45 wt %, 1-40 wt %, 10-40 wt %, 10-30 wt %, 5-10 wt %, 5-15 wt %, 5-20 wt %, 10-20 wt %, or 20-30 wt % of the bioremediation formulation.

Illustrative, non-exclusive examples of high-mobility oxidants according to the present disclosure include nitrate salts 22. Illustrative, non-exclusive examples of nitrate salts 22 according to the present disclosure include potassium nitrate, sodium nitrate, magnesium nitrate, cobalt nitrate, calcium nitrate, ammonium nitrate, ammonium phosphate nitrate, ammonia-ammonium nitrate, calcium ammonium nitrate, urea-ammonium nitrate, zinc nitrate, iron nitrate, manganese nitrate, cupric nitrate, and nitrate of soda potash.

These and other nitrate salts may be present within bioremediation formulation 10 individually or in combination. Thus, it is within the scope of the present disclosure that bioremediation formulations 10 disclosed herein, including the high-mobility oxidants included within the bioremediation formulations, may include a blend of nitrate salts. When present as a combination, or blend, of nitrate salts, each of the individual nitrate salts present within the bioremediation formulation may comprise any suitable proportion of the bioremediation formulation, including the illustrative, non-exclusive examples of high-mobility oxidant weight percentages listed above.

As an illustrative, non-exclusive example, bioremediation formulations 10 according to the present disclosure may include potassium nitrate, magnesium nitrate, and sodium nitrate and/or calcium nitrate. Magnesium ions exert minimal osmotic pressure on cell walls because they readily adhere to the soil matrix and/or are readily incorporated into biomass. In addition, magnesium ions may combine with sulfides present within the contaminated region, precipitating them from solution and decreasing unpleasant odors within any water contained therein. Potassium ions exert an osmotic pressure on cell walls that is opposite that of sodium and, when available to microbes, may increase tolerance to high concentrations of volatile fatty acids, thus improving microbe survival. In addition, sodium and/or calcium ions, when available to microbes, may improve the tolerance of the microbes to exposure to ammonia, which may be present in the bioremediation formulation and provided to the treatment zone.

Thus, the use of a blend of nitrate salts may improve water quality and/or decrease a potential for microbe growth inhibition and/or microbe mortality due to: (1) high sodium levels that may be present if sodium nitrate were used as the sole nitrate source, (2) high volatile fatty acid concentrations within the contaminated region, and/or (3) the inclusion of ammonia as a nitrogen source within the bioremediation formulation. This may provide for the use of higher overall bioremediation formulation concentrations within the treatment zone and increase a rate of contaminant consumption within the treatment zone. These bioremediation formulation concentrations, which are discussed in more detail herein, may be one or more orders of magnitude higher than the concentrations that would be permissible without utilizing a blend of nitrate salts and/or if the nitrate species disclosed herein were supplied as a nutrient for an aerobic bioremediation process as opposed to being supplied as an oxidant for anaerobic oxidative bioremediation.

Mid-mobility oxidants 80 include any suitable composition, other than molecular oxygen, that is adapted to function as an oxidant, or electron acceptor, during the anaerobic microbial respiration process and that is less mobile within the treatment zone when compared to high-mobility oxidants but more mobile within the treatment zone when compared to low-mobility oxidants. Similarly, low-mobility oxidants 40 include any suitable composition, other than molecular oxygen, that is adapted to function as an oxidant, or electron acceptor, during the anaerobic microbial respiration process, and that is less mobile within the treatment zone when compared to mid- and/or high-mobility oxidants. The mid- and/or low-mobility oxidants may comprise any suitable proportion of bioremediation formulation 10, illustrative, non-exclusive examples of which include mid- and/or low-mobility oxidants that comprise 0-70 wt % of the bioremediation formulation, optionally including 0-30 wt %, 0-40 wt %, 1-70 wt %, 1-65 wt %, 1-60 wt %, 1-40 wt %, 1-30 wt %, 10-60 wt %, 20-60 wt %, 10-30 wt %, 5-10 wt %, 5-15 wt %, 10-20 wt %, 20-30 wt %, 20-40 wt %, or 25-35 wt % of the bioremediation formulation.

Illustrative, non-exclusive examples of mid-mobility oxidants 80 and/or low-mobility oxidants 40 according to the present disclosure include sulfate salts 42. Illustrative, non-exclusive examples of sulfate salts according to the present disclosure include calcium sulfate, magnesium sulfate, ammonium sulfate, zinc sulfate, iron sulfate, manganese sulfate, cupric sulfate, ammonium phosphate sulfate, ammonium sulfate, potassium sulfate, sulfate of potash magnesia, potassium thiosulfate, potassium zinc sulfate, magnesium bisulfate, cobalt sulfate, and cobaltous potassium sulfate. These and other sulfate salts may be present within bioremediation formulation 10 individually or in combination. When present as a combination of sulfate salts, each of the individual salts present within the bioremediation formulation may comprise any suitable proportion of the bioremediation formulation, including the illustrative, non-exclusive examples of mid- and low-mobility oxidant weight percentages listed above.

In addition to providing bioremediation formulations that may include a mixture of oxidants including a range of oxidant mobilities within the treatment zone, the systems, compositions, and methods disclosed herein also may include the use of a mixture of oxidants that may provide a range of oxidant energy states. As used herein the oxidant energy state refers to the amount of energy that is available to a microorganism when utilizing the oxidant as part of its respiratory processes. In general, oxidants with higher relative energy states will be preferentially utilized by microorganisms more quickly than oxidants with lower relative energy states due to the additional energy that is available to the microorganism when the higher energy state oxidant is consumed.

Aerobic respiration, which utilizes molecular oxygen as the oxidant, provides the largest amount of energy to microbes. However, and as discussed in more detail herein, aerobic respiration may not always be feasible and/or may not provide the greatest overall bioremediation rates due to the limited availability of molecular oxygen under certain circumstances. As discussed in more detail herein, anaerobic respiration utilizes a chemical species other than molecular oxygen as the electron acceptor, or oxidant, in the respiratory process. In general, nitrate species provide more energy than metals, such as manganese (IV) or iron (III), which provide more energy than sulfate species, which provide more energy than carbon monoxide. Thus, when the bioremediation formulations 10 disclosed herein include both nitrate and sulfate salts, the nitrate salts may be consumed by the microorganisms more quickly than the sulfate salts.

As discussed in more detail herein, the use of ionic salts as an oxidant to support anaerobic microbial respiration may provide for supplying oxidants to the treatment zone at a concentration that is many orders of magnitude higher that what may be achievable when molecular oxygen is utilized as an oxidant to support aerobic microbial respiration. As an illustrative, non-exclusive example, and when the treatment zone includes water, the solubility of molecular oxygen in the water is approximately 0.0076 grams/liter (g/L) at 20° C. In contrast, the solubility of ionic salts in water may be much higher. As an illustrative, non-exclusive example, the solubility of sodium nitrate in water is approximately 876 g/L at 20° C., over five orders of magnitude higher. Other nitrate and/or sulfate salts may have solubilities of 5,000 g/L or more at 20° C. This higher oxidant solubility may provide a greater oxidant availability to the microbes present within the treatment zone, may decrease the potential for oxidant depletion within the treatment zone, and/or may provide for a decrease in the size of equipment that is utilized in the bioremediation process by decreasing a volume of material that may be moved, pumped, and/or otherwise handled as part of the bioremediation process.

In addition, and while it may be possible to supply molecular oxygen to a treatment zone through the direct delivery of oxygen and/or air to the treatment zone and/or through the injection of oxygen-releasing compounds into the treatment zone, this molecular oxygen may be consumed quickly due to its high energy state, may not remain in the treatment zone due to solubility limitations, and/or may be produced within the treatment zone at a rate that is dependent on local temperatures and pressures, as opposed to microorganism demand (as may be the case for oxygen-releasing compounds). In contrast, the oxidants disclosed herein, which take the form of ionic salts, may exist at high concentrations within the treatment zone and may persist within the treatment zone until they are consumed by microorganisms to support their respiratory processes. Thus, the systems, compositions, and methods disclosed herein may provide a more targeted and efficient oxidant delivery mechanism when compared to delivery of molecular oxygen to the treatment zone. In addition, the slower consumption of the ionic salts may decrease the potential for damage to the microbial population due to high oxidant concentrations, increasing a threshold oxidant concentration within the treatment zone above which the oxidant may damage, or inhibit the growth of, the microbial population.

In contrast with oxidants, which are utilized to accept electrons during microbial respiration, nutrient material 60 may provide a portion of the energy, or food, utilized to support microbial respiration. In general, bioremediation formulations may be designed and supplied to the treatment zone in a way that encourages, or promotes, consumption of the contaminant as a food, or energy, source for the microorganisms present therein. However, the contaminant may not include a source of nutrients that will support a target, or desired, rate and/or extent of microbe growth and/or biomass formation. Thus, it is within the scope of the present disclosure that bioremediation formulations 10 disclosed herein may include or contain any suitable additional nutrient 60 that may supplement or otherwise augment microbial respiration.

It is within the scope of the present disclosure that nutrients 60 may be selected to accomplish any suitable purpose. As an illustrative, non-exclusive example, the nutrients may be selected to increase a rate of contaminant consumption within the treatment zone. As another illustrative, non-exclusive example, the nutrients may be selected to increase a rate and/or extent of microbial biomass formation within the treatment zone. As yet another illustrative, non-exclusive example, the nutrients may be selected to encourage a certain respiratory process over another respiratory process. Illustrative, non-exclusive examples of respiratory processes according to the present disclosure include respiratory processes that form biomass and respiratory processes that produce energy for the microorganisms. As yet another illustrative, non-exclusive example, nutrients 60 may be selected to supplement, or augment, the naturally occurring nutrients that are already present within the subterranean formation. Nutrients 60 may include macronutrients, which may be utilized in large quantities by the microorganisms, as well as micronutrients, which may be utilized in relatively smaller quantities by the microorganisms. As yet another illustrative, non-exclusive example, nutrients 60 may be selected to function as a growth substrate for the microorganisms.

Illustrative, non-exclusive examples of macronutrients according to the present disclosure include nitrogen, ammonium, phosphorous, phosphate, pyrophosphate, and/or potassium. Illustrative, non-exclusive examples of micronutrients according to the present disclosure include iron, magnesium, zinc, copper, manganese, selenium, and/or B-vitamins. These nutrients may be provided from chemically and/or biologically derived sources to provide the nitrogen, phosphorus, potassium, and/or micronutrients needed to support microbial respiration at the desired, or target, growth rates. In addition to these individual nutrients, bioremediation formulations 10 according to the present disclosure may include materials that may provide a plurality of nutrients to the microorganisms. Illustrative, non-exclusive examples of materials that may provide a plurality of nutrients to the microorganisms include brewer's yeast 62 and/or complex sugars 64.

Brewer's yeast 62 may be present within a bioremediation formulation 10 in any suitable amount, proportion, or percentage, and may provide micronutrients and/or macronutrients to the microorganisms. Brewer's yeast is a type of fungus that may be used in the manufacture of bread and/or beer and/or may be obtained as a byproduct of a brewing process. As an illustrative, non-exclusive example, brewer's yeast may include yeast from the genus Saccharomyces, such as the yeast Saccharomyces cerevisiae.

Brewer's yeast is available in both active and inactive forms. In its active form, brewer's yeast may ferment carbohydrates when it comes into contact with them, forming carbon dioxide. In contrast with other yeast products, such as yeast extract, brewer's yeast includes an intact cell wall. This intact cell wall may increase the stability of the nutrients supplied by the brewer's yeast within the treatment zone, may serve as a complex sugar source for the microbes, and/or may provide for a longer residence time within the treatment zone and/or a more targeted delivery of nutrients to the microorganisms present therein. However, it is also within the scope of the present disclosure that other yeast products, including yeast extract, may be utilized with the systems, compositions, and methods disclosed herein.

Brewer's yeast, another suitable yeast product, such as yeast extract, or combinations thereof may be included within bioremediation formulation 10 in any suitable amount or proportion. As an illustrative, non-exclusive example, brewer's yeast may comprise 1-20 wt % of a bioremediation formulation, optionally comprising 2-18 wt %, 3-17 wt %, 5-15 wt %, 7-12 wt %, 5-10 wt %, or 10-15 wt % of the bioremediation formulation.

One or more complex sugars 64 also may be present in bioremediation formulations 10 according to the present disclosure in any suitable amount, proportion, or percentage. These complex sugars may improve the growth and/or maintenance of the microorganisms present within the treatment zone by acting as a microbial nutrient and/or growth substrate. Complex sugars may include various degrees of molecular branching and/or substitution and may provide a slow-release sugar source within the treatment zone. Illustrative, non-exclusive examples of complex sugars according to the present disclosure include polysaccharides, ribose, sugar-protein complexes, glycoproteins, α-bonded polysaccharides, starches, amylopectin, β-bonded polysaccharides, cellulose, carboxymethylcellulose, modified β-bonded polysaccharides, and chitin.

A specific complex sugar 64 or a plurality of complex sugars 64 may be utilized within bioremediation formulations 10 to tailor the bioavailability of the complex sugar, or sugars, and thus the rate at which the complex sugars may be consumed by microorganisms. As an illustrative, non-exclusive example, α-bonded polysaccharide molecules, such as starches, typically include some degree of branching and are readily digestible by many microorganisms. However, the digestion rate may be slowed through the use of cross-linked, α-bonded polysaccharide molecules such as pectin or amylopectin. In contrast, β-bonded polysaccharides, such as cellulose, must undergo spontaneous hydrolysis or be digested by specialized enzymes to release individual glucose molecules. This may slow the rate at which they may be consumed by the microorganisms present within the treatment zone and/or may extend their longevity within the treatment zone. Modified β-bonded polysaccharides such as chitin and carboxymethylcellulose may be consumed by microorganisms even more slowly.

Complex sugar 64 may be present within bioremediation formulations 10 in any suitable amount or proportion. As an illustrative, non-exclusive example, the complex sugar may comprise 1-20 wt % of a bioremediation formulation, optionally comprising 1-2 wt %, 1-3 wt %, 3-5 wt %, 3-7 wt %, 2-18 wt %, 3-17 wt %, 5-15 wt %, 7-12 wt %, 5-10 wt %, or 10-15 wt % of the bioremediation formulation. One or more complex sugars may be present within bioremediation formulations 10 individually or in combination. When present in combination, each of the individual complex sugars present within a bioremediation formulation may comprise any suitable proportion of the bioremediation formulation, including any of the illustrative, non-exclusive examples of complex sugar proportions listed above.

As discussed, bioremediation formulations 10 also may optionally include additional components 90, illustrative, non-exclusive examples of which include one or more phosphate salts 92, one or more surfactants 94, one or more solvents 96, and/or one or more bio-augmentation species 98. The additional components may include components that may be consumed during microbial respiration, as well as components that may increase the availability of nutrients and/or contaminants to microorganisms present within the treatment zone, such as through solvation, dissolution, and the like.

Illustrative, non-exclusive examples of phosphate salts 92, which also may be referred to herein as phosphates 92, according to the present disclosure include diammonium phosphate, ammonium phosphate, and tetrapotassium phosphate. Phosphate salt 92 may provide a source of elemental phosphorous that may be utilized by the microorganisms during respiration and may be present within the bioremediation formulation in any suitable proportion or amount. As an illustrative, non-exclusive example, the phosphate salt may comprise 1-40 wt % of the bioremediation formulation, optionally comprising 5-35 wt %, 10-30 wt %, 10-20 wt %, 20-30 wt %, 15-25 wt %, or 18-22 wt % of the bioremediation formulation. The phosphate salts may be present within a bioremediation formulation individually or in combination. When present in combination, each of the individual phosphate salts present within a bioremediation formulation may comprise any suitable proportion of the bioremediation formulation, including any of the illustrative, non-exclusive examples of phosphate salt proportions listed above.

Bioremediation formulations 10 according to the present disclosure also may include at least one surfactant 94, at least one solvent 96, at least one chemical oxidant 97, and/or at least one bio-augmentation species 98. Illustrative, non-exclusive examples of surfactants 94 according to the present disclosure include ionic surfactants and non-ionic surfactants. Surfactants, when present, may increase a water solubility of at least a portion of bioremediation formulation 10 and/or at least a portion of the contaminant, which may increase a potential for contact among the microorganisms, the bioremediation formulation, and/or the contaminant within the treatment zone and increase the rate of contaminant consumption. Solvents 96 may perform a similar function to that of surfactant 94. Illustrative, non-exclusive examples of solvents 96 according to the present disclosure include water, as well as suitable co-solvent mixtures.

Chemical oxidants 97 may directly oxidize contaminants present within the treatment zone without the need for and/or use of a microorganism as an intermediary. It is within the scope of the present disclosure that chemical oxidants 97, when present, may not be consumed by the microorganisms present within the treatment zone. However, it is also within the scope of the present disclosure that the chemical oxidants may chemically oxidize contaminants and also be consumed by the microorganisms present within the treatment zone as at least one of a nutrient and an oxidant.

As discussed in more detail herein, bio-augmentation species 98 may include microorganisms that are selected, created, and/or propagated based upon their enhanced ability to consume a particular, or target, contaminant that may be present within the treatment zone. Thus, the addition of bio-augmentation species 98 to the bioremediation formulations disclosed herein may increase a rate of consumption of the target contaminant within the treatment zone.

Any suitable criteria may be utilized to select an appropriate, desired, and/or target amount or proportion for a given component and/or group of components within a bioremediation formulation 10. This may include criteria that may be based upon site-specific conditions at the contaminated site, handling and/or storage constraints, estimated microbial nutrient demand, hydraulic characteristics of the contaminated site, geochemical characteristics of the contaminated site, geologic characteristics of the contaminated site, and/or the nature of the contaminants present within the contaminated site.

As an illustrative, non-exclusive example, a mass of the bioremediation formulation provided to the treatment zone may be calculated based, at least in part, upon a mass of contaminant present within the treatment zone and/or the contaminated region. As another illustrative, non-exclusive example, the mass of the bioremediation formulation provided to the treatment zone may be calculated based, at least in part, on a mass of contaminant that may be removed from the treatment zone to bring a concentration of contaminant within the treatment zone into compliance with regulatory requirements. This may include providing less bioremediation formulation to the treatment zone than would be needed to consume all of the contaminants that may be contained therein.

As another illustrative, non-exclusive example, the mass of bioremediation formulation provided to the treatment zone may be calculated based, at least in part, on a mass of competing electron donors that may be present within the treatment zone and/or the contaminated region. As yet another illustrative, non-exclusive example, the mass of bioremediation formulation provided to the treatment zone may be calculated based, at least in part, on the mass of the contaminant and the mass of the competing electron donors that may be present within the treatment zone and/or the contaminated region.

Illustrative, non-exclusive examples of the mass of the bioremediation formulation that may be provided to the treatment zone include masses of at least 0.2, at least 0.4, at least 0.5, at least 0.6, at least 0.7, at least 0.8, at least 1, at least 1.5, at least 2, at least 2.5, at least 3, at least 3.5, at least 4, at least 4.5, or at least 5 kilograms of the bioremediation formulation for each kilogram of the contaminant. Additionally or alternatively, the mass of the bioremediation formulation that may be provided to the treatment zone may include masses of less than 10, less than 9.5, less than 9, less than 8.5, less than 8, less than 7.5, less than 7, less than 6.5, less than 6, less than 5.5, less than 5, less than 4.5, less than 4, less than 3.5, less than 3, or less than 2.5 kilograms of the bioremediation formulation for each kilogram of the contaminant. However, values outside these ranges are also within the scope of the present disclosure.

As used herein, the phrase “competing electron donors” may refer to materials other than the contaminant that may provide electrons to, and thus consume, the oxidants present within the bioremediation formulation. Illustrative, non-exclusive examples of competing electron donors according to the present disclosure include manganese (III), iron (II), sulfide, methane, hydrogen, and/or volatile fatty acids.

The proportion of components contained within a bioremediation formulation 10 may be selected to provide a desired, specific, and/or target nitrogen to phosphorous ratio within the bioremediation formulation. This may include target nitrogen to phosphorous ratios of between 2:1 and 8:1, such as target nitrogen to phosphorous ratios between 3:1 and 6:1, as well as nitrogen to phosphorous ratios of approximately 3.5:1, 4:1, 4.5:1, 5:1, or 5.5:1. Other ratios, or proportions, are also within the scope of the present disclosure. Higher phosphorous concentrations may be utilized with the systems, compositions, and methods disclosed herein when compared to more traditional bioremediation formulations, which may include a nitrogen to phosphorous ratio of approximately 10:1, to encourage the formation of microorganism biomass within the treatment zone.

Bioremediation formulations 10 disclosed herein may be created, stored, and/or supplied to the treatment zone in any suitable form. As an illustrative, non-exclusive example, the bioremediation formulations may include dry bioremediation formulations. Illustrative, non-exclusive examples of dry bioremediation formulations include powdered, granular, and/or pellet forms. These dry bioremediation formulations may include substantially heterogeneous or substantially homogeneous mixtures. When a bioremediation formulation 10 includes a dry bioremediation formulation, it is within the scope of the present disclosure that the dry bioremediation formulation may include a coating or other time-release structure adapted or configured to control a rate of delivery of the bioremediation formulation to the treatment zone.

Bioremediation formulations 10 disclosed herein also may include solutions of the bioremediation formulations. An illustrative, non-exclusive example of a solution of a bioremediation formulation according to the present disclosure includes an aqueous solution of the bioremediation formulation. When the bioremediation formulation is included in an aqueous solution, the aqueous solution may include any suitable amount or proportion of the bioremediation formulation. As an illustrative, non-exclusive example, the aqueous solution may include 5-65 wt % of the bioremediation formulation, including 5-50 wt %, 5-40 wt %, 5-30 wt %, 5-20 wt %, 5-15 wt %, 5-10 wt %, 10-25 wt %, 10-20 wt %, 10-15 wt %, approximately 11 wt %, approximately 12 wt %, approximately 13 wt %, approximately 14 wt %, or approximately 15 wt % of the bioremediation formulation. Other weight percentages are also within the scope of the present disclosure.

FIG. 2 provides an illustrative, non-exclusive example of a contaminated region 100 according to the present disclosure. Contaminated region 100 may include or contain a contaminant 102 and may be associated with one or more treatment zones 120. As used herein, the phrase “associated with” means that at least a portion of treatment zone 120 may be proximal to, in fluid communication with, in physical contact with, contained within, and/or contain at least a portion of contaminated region 100. The contaminated region also may include or contain one or more microorganisms 104 that may be adapted to consume contaminant 102 as part of their natural respiratory processes.

It is within the scope of the present disclosure that treatment zone 120 may be associated with contaminated region 100 in any suitable manner. As an illustrative, non-exclusive example, and with reference to FIG. 2, treatment zone 120 may include and/or contain all of contaminated region 100 as shown at 121. As another illustrative, non-exclusive example, treatment zone 120 may be completely included and/or contained within contaminated region 100 as shown at 122. As yet another illustrative, non-exclusive example, treatment zone 120 may be coextensive with contaminated region 100 as shown at 123. As yet another illustrative, non-exclusive example, treatment zone 120 may include and/or contain a portion of contaminated region 100 as shown at 124. As yet another illustrative, non-exclusive example, treatment zone 120 may be proximal to and/or form a barrier around at least a portion of contaminated region 100 as shown at 125. As yet another illustrative, non-exclusive example, treatment zone 120 may be adjacent to at least a portion of contaminated region 100 as shown at 126.

When the treatment zone is proximal to and/or forms a barrier around at least a portion of the contaminated region as shown at 125 and/or when the treatment zone is adjacent to the contaminated region as shown at 126, the treatment zone may be located to decrease a migration of contaminants away from contaminated region 100 and/or to increase treatment of contaminated region 100. As an illustrative, non-exclusive example, and when there is a flow of water, such as groundwater, through the contaminated region, treatment zone 120 may be located downstream of the contaminated region and may serve as a permeable reactive barrier to decrease the potential for contaminant migration away from the contaminated region. Additionally or alternatively, treatment zone 120 may be located upstream of the contaminated region, may mix with the flow of water, and may serve to provide a supply of bioremediation formulation to the contaminated region as the bioremediation formulation is carried from the treatment zone to the contaminated region by the flow of water, expanding the size of the treatment zone.

It is within the scope of the present disclosure that the bioremediation formulations disclosed herein may be present within and/or applied to the treatment zone and/or the contaminated region in any suitable manner. As an illustrative, non-exclusive example, bioremediation formulations 10 may be uniformly, or substantially uniformly, distributed throughout the treatment zone. Bioremediation formulations 10 that are uniformly distributed throughout the treatment zone may be uniformly distributed at the time of introduction into the treatment zone, such as by being uniformly mixed into the treatment zone. Additionally or alternatively, the bioremediation formulations may be uniformly distributed over time by diffusion and/or fluid flows within the treatment zone.

Bioremediation formulations 10 also may exhibit a concentration gradient across the treatment zone. As an illustrative, non-exclusive example, and with continued reference to FIG. 2, a bioremediation formulation 10 may be initially applied to the treatment zone at one or more application sites 140. The bioremediation formulation may then diffuse, flow, or otherwise move from the initial application site with time. This movement may be based, at least in part, on the mobility of the individual components that comprise the bioremediation formulation, as discussed in more detail herein.

Thus, it is within the scope of the present disclosure that, when a bioremediation formulation 10 includes a high-mobility oxidant 20 and a low-mobility oxidant 40, the high-mobility oxidant may move away from the application site more quickly than the low-mobility oxidant, increasing the overall size of a treatment zone 120. Similarly, the low-mobility oxidant may diffuse more slowly than the high-mobility oxidant and/or may remain within, or substantially within, the application site. Thus, bioremediation formulations 10 according to the present disclosure may provide directed, or controlled, oxidant availability over a variety of length scales, or distances, within the treatment zone.

Contaminated region 100 may include any suitable region or structure that may include or contain contaminant 102. Illustrative, non-exclusive examples of contaminated regions according to the present disclosure include aquifers, lakes, rivers, streams, soil samples, fields, parking areas, industrial sites, commercial sites, waste disposal sites, and/or landfills. These contaminated regions include contaminant 102 and also may include liquids, such as water, as well as solids, such as biomass, soil, and/or rocks.

Bioremediation formulation 10 may be supplied to treatment zone 120 using any suitable method or mechanism. As an illustrative, non-exclusive example, and when contaminated region 100 includes an aquifer, lake, river, stream, and/or another site that includes water, bioremediation formulation 10 may be supplied to the treatment zone as an aqueous solution. This may include slug injecting concentrated aqueous solutions of the bioremediation formulation using any suitable well, monitoring well, injection well, infiltration gallery, direct push technology, lance injection technology, and/or push probe. Additionally or alternatively, it is within the scope of the present disclosure that the aqueous bioremediation formulation may be supplied to the treatment zone as part of a groundwater recirculation treatment in which groundwater may be pumped from a suitable subsurface region, combined with the bioremediation formulation, and returned to the treatment zone. It is also within the scope of the present disclosure that the bioremediation formulations disclosed herein may be utilized to provide oxidative treatment of the treatment zone during air sparging applications in which air, molecular oxygen, ozone, or other gaseous oxidants are injected directly into the treatment zone and/or the groundwater.

As another illustrative, non-exclusive example, and when contaminated region 100 includes a soil sample, field, parking area, industrial site, commercial site, waste disposal site, or other surface or near-surface region, bioremediation formulation 10 may be applied to the treatment zone as an aqueous solution and/or as a dry bioremediation formulation, including any of the dry bioremediation formulations disclosed herein. When the bioremediation formulation is applied to the treatment zone as an aqueous solution, it may be sprayed, injected, irrigated, flooded, and/or chemigated onto the treatment zone. When the bioremediation formulation is applied to the treatment zone as a dry bioremediation formulation, it may be spread onto, broadcast onto, and/or mixed into the treatment zone.

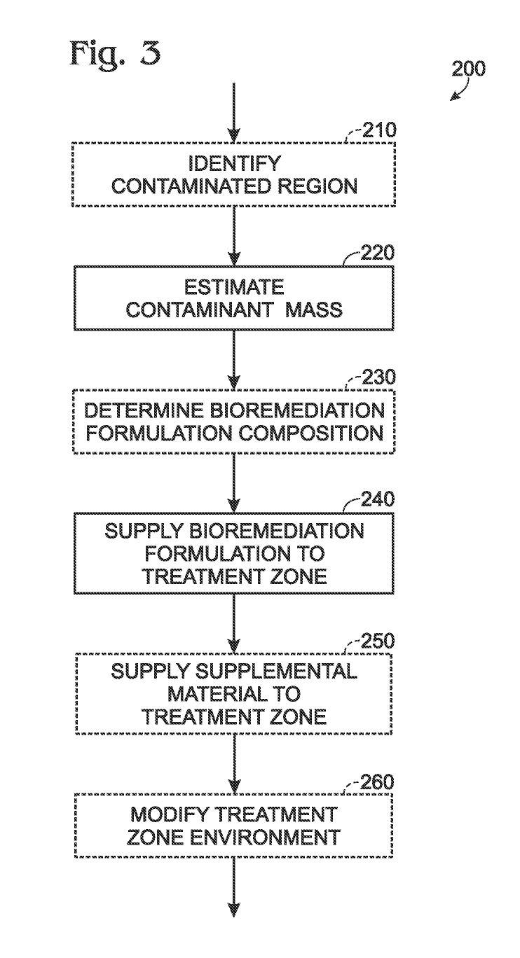

FIG. 3 provides illustrative, non-exclusive examples of methods 200 of supplying a bioremediation formulation 10 to a treatment zone 120. Methods 200 may include identifying a contaminated region at 210 and include estimating a mass of contaminant contained within the contaminated region at 220. Methods 200 also may include determining a desired composition of a bioremediation formulation 10 at 230 and include supplying the bioremediation formulation to a treatment zone at 240. Methods 200 further may include supplying supplemental materials to the treatment zone at 250 and/or modifying the treatment zone environment at 260.

Identifying the contaminated region at 210 may include the use of any suitable method, procedure, detector, test, monitor, historical information, and/or observation to identify a contaminated region 100. It is within the scope of the present disclosure that identifying the contaminated region at 210 may include at least identifying the general location of the contaminated region.

However, it is also within the scope of the present disclosure that identifying the contaminated region also may include testing to determine the nature of the contaminants that are present within the contaminated region, the chemical composition of the contaminants that are present within the contaminated region, the extent of the contaminated region, the nature of the materials contained within the contaminated region, the mass of competing electron donors that may be present within the contaminated region, the volume of the contaminated region, the surface area of the contaminated region, the depth of the contaminated region, the geological conditions of the contaminated region, the hydrogeological conditions of the contaminated region, the soil type within the contaminated region, the organic content within the contaminated region, the contaminant mobility within the contaminated region, the groundwater flow direction within the contaminated region, the groundwater flow velocity within the contaminated region, the background, or native, electron acceptor concentration within the contaminated region, and/or the identity of and/or the metabolic processes utilized by microorganisms 104 that are present within the contaminated region. It is further within the scope of the present disclosure that identifying the contaminated region may include determining any suitable characteristic and/or property of the contaminated region, including the pH of the contaminated region, the oxygen content of the contaminated region, the water content of the contaminated region, the permeability of the contaminated region, the structure of the native strata contained within the contaminated region, the affinity of contaminant 102 for the native strata contained within the contaminated region, the mobility of contaminant 102 within the contaminated region, and/or the mobility and/or potential mobility of one or more components of bioremediation formulation 10 within the contaminated region.

Estimating the mass of contaminant contained within the contaminated region at 220 may include utilizing any suitable and/or available information to estimate, approximate, or measure the mass of contaminant contained within the contaminated region. This may include the use of any of the information about the contaminated region that is discussed herein and may further include measuring a concentration, mass, and/or amount of contaminant in one or more portions of, and/or samples that are taken from, the contaminated region, calculating the mass of contaminant utilizing one or more partitioning coefficients, measuring a mass of organic carbon in the contaminated region, and/or knowledge of a known mass of contaminant that may have been released into the contaminated region.

It is within the scope of the present disclosure that the contaminated region may include a single contaminant or a plurality of contaminants. When the contaminated region includes a single contaminant, estimating the mass of contaminant contained within the contaminated region may include estimating the mass of the single contaminant. When the contaminated region includes a plurality of contaminants, estimating the mass of contaminant contained within the contaminated region may include estimating the total mass of contaminant and/or estimating the mass of one or more individual contaminants contained within the contaminated region.

Determining the bioremediation formulation composition at 230 may include the use of any suitable criteria to determine a suitable bioremediation formulation 10 for use in a given treatment zone. As an illustrative, non-exclusive example, this may include selecting, or determining, the bioremediation formulation composition based at least in part on at least one of a characteristic of the treatment zone, a characteristic of the contaminated region, a characteristic of the contaminant, a mobility of the bioremediation formulation within the treatment zone, and/or a characteristic of the native microbe population. Illustrative, non-exclusive examples of characteristics of the contaminated region may include any suitable characteristic determined when identifying the contaminated region at 210.

Illustrative, non-exclusive examples of characteristics of the contaminant may include any suitable characteristic of the contaminant, including the characteristics discussed herein. Illustrative, non-exclusive examples of characteristics of the native microbe population include any of the characteristics disclosed herein, including the identity of one or more microbes included in the native microbe population, the metabolic processes performed by one or more microbes included in the native microbe population, a rate at which one or more contaminants may be consumed by one or more microbes present in the native microbe population, and/or target environmental conditions for improved growth and/or metabolic functioning of one or more microbes included in the native microbe population.

Supplying the bioremediation formulation to the treatment zone at 240 may include supplying a mass of the bioremediation formulation to one or more treatment zones. It is within the scope of the present disclosure that supplying the bioremediation formulation to the treatment zone may include supplying the bioremediation formulation based at least in part on the mass of contaminant within the contaminated region as estimated at 220.

As an illustrative, non-exclusive example, and as discussed in more detail herein, this may include supplying a mass of bioremediation formulation that is calculated based at least in part on the calculated mass of contaminant. As another illustrative, non-exclusive example, this may include supplying a mass of bioremediation formulation such that, subsequent to consumption of the contaminant by the microorganisms, less than 30%, less than 25%, less than 20%, less than 15%, less than 10%, less than 5%, less than 2.5%, less than 1%, or less than 0.5% of the bioremediation formulation, and/or of any suitable component thereof, remains within the treatment zone. As another illustrative, non-exclusive example, the supplying may include determining a hydrogen equivalent for the contaminant, each contaminant of the plurality of contaminants, and/or the competing electron donors that may be present within the contaminated region and/or the treatment zone, calculating a total hydrogen equivalent based thereon, and/or calculating the mass of bioremediation formulation to be provided to the treatment zone based, at least in part, on the calculated total hydrogen equivalent of the contaminant, or plurality of contaminants.

As discussed in more detail herein, supplying the bioremediation formulation to the treatment zone at 240 may include supplying the bioremediation formulation in any suitable form, illustrative, non-exclusive examples of which include supplying the bioremediation formulation in an aqueous solution and supplying the bioremediation formulation as a dry bioremediation formulation. As discussed in more detail herein, when the bioremediation formulation is supplied to the treatment zone as an aqueous solution, the bioremediation formulation may be injected into the treatment zone using any suitable direct push technology, lance injection technique, push probe, monitoring well, and/or infiltration gallery. Similarly, and when the bioremediation formulation is supplied to the treatment zone as a dry bioremediation formulation, the bioremediation formulation may be applied to and/or mixed with the materials that comprise the contaminated region, such as the contaminated soil within the contaminated region.

It is within the scope of the present disclosure that supplying the bioremediation formulation to the treatment zone may include contacting the bioremediation formulation with the contaminant, surrounding at least a portion of the contaminated region with the bioremediation formulation, and/or flowing the bioremediation formulation into the contaminated region. As an illustrative, non-exclusive example, and as discussed in more detail herein, flowing the bioremediation formulation into the contaminated region may include producing groundwater, mixing the bioremediation formulation with the produced groundwater to produce an aqueous bioremediation formulation, and supplying the aqueous bioremediation formulation to the treatment zone with the produced, or recirculated, groundwater.

It is also within the scope of the present disclosure that supplying the bioremediation formulation to the treatment zone may include supplying the bioremediation formulation at any suitable rate and/or using any suitable process. As an illustrative, non-exclusive example, and when the contaminated region includes a high flow rate aquifer, the supplying may include repeatedly and/or periodically supplying the bioremediation formulation to the treatment zone. The periodically supplying may decrease a potential for the bioremediation formulation to flow out of the contaminated region and/or provide time for the microbes that are present within the treatment zone to utilize the bioremediation formulation during their metabolic process, thus increasing the biomass of microbes that are present within the treatment zone.

Supplying supplemental material to the treatment zone at 250 may include supplying any suitable material to complement and/or in addition to the bioremediation formulation. As an illustrative, non-exclusive example, this may include providing an air stream to the treatment zone. As another illustrative, non-exclusive example, this may include providing one or more chemical oxidants to the treatment zone to chemically oxidize at least a portion of the contaminants contained within the contaminated region.

Modifying the treatment zone environment at 260 may include the use of any suitable material, method, and/or process to modify the treatment zone environment and/or promote the anaerobic microbial bioremediation of contaminants contained therein. As an illustrative, non-exclusive example, this may include at least one of creating an environment in which the bioremediation formulation is consumed as an oxidant by the microbes present within the treatment zone, creating an environment in which the contaminant is consumed during anaerobic microbial respiration, and/or creating an environment in which the contaminant is oxidized during the anaerobic microbial respiration. Illustrative, non-exclusive examples of modifying the treatment zone environment include changing the pH of the treatment zone, changing the temperature of the treatment zone, changing the oxygen content of the treatment zone, changing the concentration of one or more chemical compositions or elements within the treatment zone, changing the flow characteristics of fluids contained within the treatment zone, changing the permeability of at least a portion of the treatment zone, and/or changing the identity and/or concentration of microbes present within the treatment zone.

In the present disclosure, several of the illustrative, non-exclusive examples of methods have been discussed and/or presented in the context of flow diagrams, or flow charts, in which the methods are shown and described as a series of blocks, or steps. Unless specifically set forth in the accompanying description, it is within the scope of the present disclosure that the order of the blocks may vary from the illustrated order in the flow diagram, including with two or more of the blocks (or steps) occurring in a different order and/or concurrently. It is also within the scope of the present disclosure that the blocks, or steps, may be implemented as logic, which also may be described as implementing the blocks, or steps, as logics. In some applications, the blocks, or steps, may represent expressions and/or actions to be performed by functionally equivalent circuits or other logic devices. The illustrated blocks may, but are not required to, represent executable instructions that cause a computer, processor, and/or other logic device to respond, to perform an action, to change states, to generate an output or display, and/or to make decisions.

As used herein, the term “and/or” placed between a first entity and a second entity means one of (1) the first entity, (2) the second entity, and (3) the first entity and the second entity. Multiple entities listed with “and/or” should be construed in the same manner, i.e., “one or more” of the entities so conjoined. Other entities may optionally be present other than the entities specifically identified by the “and/or” clause, whether related or unrelated to those entities specifically identified. Thus, as a non-limiting example, a reference to “A and/or B,” when used in conjunction with open-ended language such as “comprising” may refer, in one embodiment, to A only (optionally including entities other than B); in another embodiment, to B only (optionally including entities other than A); in yet another embodiment, to both A and B (optionally including other entities). These entities may refer to elements, actions, structures, steps, operations, values, and the like.

As used herein, the phrase “at least one,” in reference to a list of one or more entities should be understood to mean at least one entity selected from any one or more of the entity in the list of entities, but not necessarily including at least one of each and every entity specifically listed within the list of entities and not excluding any combinations of entities in the list of entities. This definition also allows that entities may optionally be present other than the entities specifically identified within the list of entities to which the phrase “at least one” refers, whether related or unrelated to those entities specifically identified. Thus, as a non-limiting example, “at least one of A and B” (or, equivalently, “at least one of A or B,” or, equivalently “at least one of A and/or B”) may refer, in one embodiment, to at least one, optionally including more than one, A, with no B present (and optionally including entities other than B); in another embodiment, to at least one, optionally including more than one, B, with no A present (and optionally including entities other than A); in yet another embodiment, to at least one, optionally including more than one, A, and at least one, optionally including more than one, B (and optionally including other entities). In other words, the phrases “at least one,” “one or more,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C” and “A, B, and/or C” may mean A alone, B alone, C alone, A and B together, A and C together, B and C together, A, B and C together, and optionally any of the above in combination with at least one other entity.

In the event that any patents, patent applications, or other references are incorporated by reference herein and define a term in a manner or are otherwise inconsistent with either the non-incorporated portion of the present disclosure or with any of the other incorporated references, the non-incorporated portion of the present disclosure shall control, and the term or incorporated disclosure therein shall only control with respect to the reference in which the term is defined and/or the incorporated disclosure was originally present.

As used herein the terms “adapted” and “configured” mean that the element, component, or other subject matter is designed and/or intended to perform a given function. Thus, the use of the terms “adapted” and “configured” should not be construed to mean that a given element, component, or other subject matter is simply “capable of” performing a given function but that the element, component, and/or other subject matter is specifically selected, created, implemented, utilized, programmed, and/or designed for the purpose of performing the function. It is also within the scope of the present disclosure that elements, components, and/or other recited subject matter that is recited as being adapted to perform a particular function may additionally or alternatively be described as being configured to perform that function, and vice versa.

Illustrative, non-exclusive examples of systems and methods according to the present disclosure are presented in the following enumerated paragraphs. It is within the scope of the present disclosure that an individual step of a method recited herein, including in the following enumerated paragraphs, may additionally or alternatively be referred to as a “step for” performing the recited action.

A1. A bioremediation formulation configured to provide at least a portion of the oxidants and nutrients that are consumed by a native microbe population during anaerobic respiration and to promote anaerobic oxidative bioremediation of a contaminant contained within a treatment zone that is associated with a contaminated region, the bioremediation formulation comprising:

a high-mobility oxidant, wherein the high-mobility oxidant has a high-mobility oxidant diffusion constant when present within the treatment zone;

a low-mobility oxidant, wherein the low-mobility oxidant has a low-mobility oxidant diffusion constant when present within the treatment zone, and further wherein the low-mobility oxidant diffusion constant is less than the high-mobility oxidant diffusion constant; and

a nutrient material.

A2. The bioremediation formulation of paragraph A1, wherein the bioremediation formulation further includes a mid-mobility oxidant, wherein the mid-mobility oxidant has a mid-mobility oxidant diffusion constant when present within the treatment zone, wherein the mid-mobility oxidant diffusion constant is less than the high-mobility oxidant diffusion constant, and further wherein the mid-mobility oxidant diffusion constant is greater than the low-mobility oxidant diffusion constant.

A3. The bioremediation formulation of paragraph A2, wherein the mid-mobility oxidant comprises 1-70 wt % of the bioremediation formulation, optionally including 1-65 wt %, 1-60 wt %, 1-40 wt %, 1-30 wt %, 10-60 wt %, 20-60 wt %, 10-30 wt %, 5-10 wt %, 5-15 wt %, 10-20 wt %, 20-30 wt %, 20-40 wt %, or 25-35 wt % of the bioremediation formulation, and further optionally wherein the wt % includes a wt % of the active components of the bioremediation formulation.

A4. The bioremediation formulation of any of paragraphs A2-A3, wherein the mid-mobility oxidant includes a sulfate salt.

A5. The bioremediation formulation of paragraph A4, wherein the sulfate salt includes at least one of calcium sulfate, magnesium sulfate, and ammonium sulfate.

A6. The bioremediation formulation of any of paragraphs A1-A5, wherein the nutrient material includes at least a first complex sugar.

A7. The bioremediation formulation of paragraph A6, wherein the at least a first complex sugar includes at least one of an α-bonded polysaccharide, a starch, an amylopectin, a β-bonded polysaccharide, cellulose, a modified β-bonded polysaccharide, chitin, carboxymethylcellulose, ribose, and a glycoprotein.

A8. The bioremediation formulation of any of paragraphs A6-A7, wherein the at least a first complex sugar comprises 1-20 wt % of the bioremediation formulation, optionally comprising 2-18 wt %, 3-17 wt %, 5-15 wt %, 7-12 wt %, 5-10 wt %, or 10-15 wt % of the bioremediation formulation, and further optionally wherein the wt % includes a wt % of the active components of the bioremediation formulation.

A9. The bioremediation formulation of any of paragraphs A1-A8, wherein the nutrient material includes brewer's yeast.

A10. The bioremediation formulation of any of paragraphs A1-A9, wherein the brewer's yeast comprises 1-20 wt % of the bioremediation formulation, optionally comprising 2-18 wt %, 3-17 wt %, 5-15 wt %, 7-12 wt %, 5-10 wt %, or 10-15 wt % of the bioremediation formulation, and further optionally wherein the wt % includes a wt % of the active components of the bioremediation formulation.

A11. The bioremediation formulation of any of paragraphs A1-A10, wherein the bioremediation formulation further includes at least a first additional component.

A12. The bioremediation formulation of paragraph A11, wherein the at least a first additional component includes at least one of an ionic surfactant, a non-ionic surfactant, a co-solvent, and a bio-augmentation species.

A13. The bioremediation formulation of any of paragraphs A1-A12, wherein the high-mobility oxidant comprises 1-50 wt % of the bioremediation formulation, optionally including 1-45 wt %, 1-40 wt %, 10-40 wt %, 10-30 wt %, 5-10 wt %, 5-15 wt %, 10-20 wt %, or 20-30 wt % of the bioremediation formulation, and further optionally wherein the wt % includes a wt % of the active components of the bioremediation formulation.

A14. The bioremediation formulation of any of paragraphs A1-A13, wherein the high-mobility oxidant includes a nitrate salt.

A15. The bioremediation formulation of paragraph A14, wherein the nitrate salt includes at least one of potassium nitrate, sodium nitrate, and magnesium nitrate.

A16. The bioremediation formulation of any of paragraphs A1-A15, wherein the low-mobility oxidant comprises 1-70 wt % of the bioremediation formulation, optionally including 1-65 wt %, 1-60 wt %, 1-40 wt %, 1-30 wt %, 10-60 wt %, 20-60 wt %, 10-30 wt %, 5-10 wt %, 5-15 wt %, 10-20 wt %, 20-30 wt %, 20-40 wt %, or 25-35 wt % of the bioremediation formulation, and further optionally wherein the wt % includes a wt % of the active components of the bioremediation formulation.

A17. The bioremediation formulation of any of paragraphs A1-A16, wherein the low-mobility oxidant includes a sulfate salt.

A18. The bioremediation formulation of paragraph A17, wherein the sulfate salt includes at least one of calcium sulfate, magnesium sulfate, and ammonium sulfate.

A19. The bioremediation formulation of any of paragraphs A1-A18, wherein the bioremediation formulation further includes at least a first phosphate, and optionally wherein the at least a first phosphate includes at least one of diammonium phosphate, ammonium polyphosphate, and tetrapotassium pyrophosphate.

A20. The bioremediation formulation of paragraph A19, wherein the at least a first phosphate comprises 1-40 wt % of the bioremediation formulation, optionally comprising 5-35 wt %, 10-30 wt %, 10-20 wt %, 20-30 wt %, 15-25 wt %, or 18-22 wt % of the bioremediation formulation, and further optionally wherein the wt % includes a wt % of the active components of the bioremediation formulation.

A21. The bioremediation formulation of any of paragraphs A19-A20, wherein a nitrogen to phosphorous ratio in the bioremediation formulation is between 2:1 and 8:1, optionally including nitrogen to phosphorous ratios between 3:1 and 6:1, and further optionally including nitrogen to phosphorous ratios of approximately 3.5:1, 4:1, 4.5:1, 5:1, or 5.5:1.

A22. The bioremediation formulation of any of paragraphs A1-A21, wherein the contaminant includes at least one of a hydrocarbon, a petroleum hydrocarbon, a metal, a partially halogenated solvent, and vinyl chloride.

A23. The bioremediation formulation of any of paragraphs A1-A22, wherein the native microbe population includes at least one of bacteria, fungi, denitrifiers, sulfate reducers, anaerobic species, facultative anaerobic species, and facultative aerobic species.

B1. A bioremediation formulation configured to provide at least a portion of the oxidants and nutrients that are consumed by a native microbe population during anaerobic respiration and to promote anaerobic oxidative bioremediation of a contaminant contained within a treatment zone that is associated with a contaminated region, the bioremediation formulation comprising:

a sulfate salt, wherein the sulfate salt comprises 20-60 wt % of the bioremediation formulation, and optionally wherein the wt % includes a wt % of the active components of the bioremediation formulation;

a nitrate salt, wherein the nitrate salt comprises 10-40 wt % of the bioremediation formulation, and optionally wherein the wt % includes a wt % of the active components of the bioremediation formulation;

a phosphate salt, wherein the phosphate salt comprises 10-30 wt % of the bioremediation formulation, and optionally wherein the wt % includes a wt % of the active components of the bioremediation formulation;

a complex sugar, wherein the complex sugar comprises 5-15 wt % of the bioremediation formulation, and optionally wherein the wt % includes a wt % of the active components of the bioremediation formulation; and

brewer's yeast, wherein the brewer's yeast comprises 5-15 wt % of the bioremediation formulation, and optionally wherein the wt % includes a wt % of the active components of the bioremediation formulation.

C1. A bioremediation formulation configured to provide at least a portion of the oxidants and nutrients that are consumed by a native microbe population during anaerobic respiration and to promote anaerobic oxidative bioremediation of a contaminant contained within a treatment zone that is associated with a contaminated region, the bioremediation formulation consisting essentially of:

a sulfate salt, wherein the sulfate salt comprises 20-60 wt % of the bioremediation formulation, and optionally wherein the wt % includes a wt % of the active components of the bioremediation formulation;

a nitrate salt, wherein the nitrate salt comprises 10-40 wt % of the bioremediation formulation, and optionally wherein the wt % includes a wt % of the active components of the bioremediation formulation;

a phosphate salt, wherein the phosphate salt comprises 10-30 wt % of the bioremediation formulation, and optionally wherein the wt % includes a wt % of the active components of the bioremediation formulation;

a complex sugar, wherein the complex sugar comprises 5-15 wt % of the bioremediation formulation, and optionally wherein the wt % includes a wt % of the active components of the bioremediation formulation; and

brewer's yeast, wherein the brewer's yeast comprises 5-15 wt % of the bioremediation formulation, and optionally wherein the wt % includes a wt % of the active components of the bioremediation formulation.

D1. The bioremediation formulation of any of paragraphs B1-C1, wherein the sulfate salt includes calcium sulfate, and optionally wherein the sulfate salt further includes at least one of magnesium sulfate and ammonium sulfate.

D2. The bioremediation formulation of any of paragraphs B1-D1, wherein the nitrate salt includes potassium nitrate, and optionally wherein the nitrate salt further includes at least one of sodium nitrate and magnesium nitrate.

D3. The bioremediation formulation of any of paragraphs B1-D2, wherein the phosphate salt includes at least one of diammonium phosphate, ammonium phosphate, and tetrapotassium pyrophosphate.

D4. The bioremediation formulation of any of paragraphs B1-D3, wherein the complex sugar includes at least one of an α-bonded polysaccharide, a starch, an amylopectin, a β-bonded polysaccharide, cellulose, a modified β-bonded polysaccharide, chitin, carboxymethylcellulose, ribose, and a glycoprotein.

E1. An aqueous bioremediation solution, the solution comprising:

water; and

the bioremediation formulation of any of paragraphs A1-D4.

E2. The aqueous bioremediation solution of paragraph E1, wherein the bioremediation formulation comprises 5-65 wt % of the aqueous bioremediation solution, optionally including 5-50 wt %, 5-40 wt %, 5-30 wt %, 5-20 wt %, 5-15 wt %, 5-10 wt %, 10-25 wt %, 10-20 wt %, 10-15 wt %, 11 wt %, 12 wt %, 13 wt %, or 14 wt % of the aqueous bioremediation solution.

F1. An aquifer comprising:

water;

a contaminant; and

the bioremediation formulation of any of paragraphs A1-D4.

F2. The aquifer of paragraph F1, wherein the contaminant includes a hydrocarbon, and further wherein the aquifer includes 0.2-10 kilograms of the bioremediation formulation per kilogram of the contaminant present within the aquifer, optionally wherein the aquifer includes at least 0.2, at least 0.4, at least 0.5, at least 0.6, at least 0.7, at least 0.8, at least 1, at least 1.5, at least 2, at least 2.5, at least 3, at least 3.5, at least 4, at least 4.5, or at least 5 kilograms of the bioremediation formulation for each kilogram of the contaminant present within the aquifer, and further optionally wherein the aquifer includes less than 10, less than 9.5, less than 9, less than 8.5, less than 8, less than 7.5, less than 7, less than 6.5, less than 6, less than 5.5, less than 5, less than 4.5, less than 4, less than 3.5, less than 3, or less than 2.5 kilograms of the bioremediation formulation for each kilogram of the contaminant present within the aquifer.

F3. The aquifer of any of paragraphs F1-F2, wherein the bioremediation formulation is present within at least one of the entire aquifer, a contaminated portion of the aquifer, and a barrier region within the aquifer.

G1. A soil sample comprising:

a contaminant; and

the bioremediation formulation of any of paragraphs A1-D4.

G2. The soil sample of paragraph G1, wherein the contaminant includes a hydrocarbon, and further wherein the soil sample includes 0.2-10 kilograms of the bioremediation formulation per kilogram of the contaminant present within the soil sample, optionally wherein the soil sample includes at least 0.2, at least 0.4, at least 0.5, at least 0.6, at least 0.7, at least 0.8, at least 1, at least 1.5, at least 2, at least 2.5, at least 3, at least 3.5, at least 4, at least 4.5, or at least 5 kilograms of the bioremediation formulation for each kilogram of the contaminant present within the soil sample, and further optionally wherein the soil sample includes less than 10, less than 9.5, less than 9, less than 8.5, less than 8, less than 7.5, less than 7, less than 6.5, less than 6, less than 5.5, less than 5, less than 4.5, less than 4, less than 3.5, less than 3, or less than 2.5 kilograms of the bioremediation formulation for each kilogram of the contaminant present within the soil sample.

G3. The soil sample of any of paragraphs G1-G2, wherein the bioremediation formulation is present within the treatment zone, and further wherein the treatment zone includes at least one of the entire soil sample, a contaminated portion of the soil sample, and a barrier region within the soil sample.

H1. A method of supplying an oxidant and nutrients to a native microbe population to promote consumption of a contaminant by anaerobic microbial respiration, wherein the contaminant is contained within a treatment zone that is associated with a contaminated region, the method comprising:

estimating a mass of contaminant present within at least a portion of the contaminated region; and

supplying a mass of the bioremediation formulation of any of paragraphs A1-D4 to the treatment zone, wherein the supplying is based at least in part on the estimating, and further wherein the supplying supports the anaerobic oxidative bioremediation of the contaminant.

H2. The method of paragraph H1, wherein the supplying includes supplying between 0.2 and 10 kilograms of bioremediation formulation per kilogram of contaminant present within the portion of the contaminated region, optionally including supplying at least 0.2, at least 0.4, at least 0.5, at least 0.6, at least 0.7, at least 0.8, at least 1, at least 1.5, at least 2, at least 2.5, at least 3, at least 3.5, at least 4, at least 4.5, or at least 5 kilograms of the bioremediation formulation for each kilogram of the contaminant present within the portion of the contaminated region, and further optionally including supplying less than 10, less than 9.5, less than 9, less than 8.5, less than 8, less than 7.5, less than 7, less than 6.5, less than 6, less than 5.5, less than 5, less than 4.5, less than 4, less than 3.5, less than 3, or less than 2.5 kilograms of the bioremediation formulation for each kilogram of the contaminant present within the portion of the contaminated region.

H3. The method of any of paragraphs H1-H2, wherein the method further includes mixing the bioremediation formulation with water to form an aqueous bioremediation solution, wherein the bioremediation formulation comprises 5-65 wt % of the aqueous bioremediation solution, optionally including 5-50 wt %, 5-40 wt %, 5-30 wt %, 5-20 wt %, 5-15 wt %, 5-10 wt %, 10-25 wt %, 10-20 wt %, 10-15 wt %, 11 wt %, 12 wt %, 13 wt %, or 14 wt % of the aqueous bioremediation solution, and further wherein the supplying includes supplying the aqueous bioremediation solution to the treatment zone, and optionally wherein the wt % includes a wt % of the active components of the bioremediation formulation.

H4. The method of paragraph H3, wherein the contaminated region includes an aquifer.

H5. The method of any of paragraphs H1-H4, wherein the method further includes providing a chemical oxidant to the treatment zone and oxidizing the contaminant with the chemical oxidant, and optionally wherein the chemical oxidant is not consumed by the native microbe population.

H6. The method of any of paragraphs H1-H5, wherein the supplying includes mixing the bioremediation formulation with a contaminated soil sample.

H7. The method of any of paragraphs H1-H6, wherein the supplying includes injecting the bioremediation formulation into the treatment zone, and optionally wherein the injecting includes at least one of injecting with a direct push technology, a lance injection technique, a push probe, injecting into a monitoring well, and injecting into an infiltration gallery.

H8. The method of any of paragraphs H1-H7, wherein the supplying includes contacting the bioremediation formulation with the contaminant.

H9. The method of any of paragraphs H1-H8, wherein the supplying includes surrounding at least a portion of the contaminated region with the bioremediation formulation.

H10. The method of any of paragraphs H1-H9, wherein the method further includes producing groundwater from the treatment zone and recirculating the produced groundwater into the treatment zone as recirculated groundwater, and optionally wherein the supplying includes supplying the bioremediation formulation in the recirculated groundwater.

H11. The method of any of paragraphs H1-H10, wherein the method further includes providing air to the treatment zone.

H12. The method of any of paragraphs H1-H11, wherein the method further includes creating an environment in which the bioremediation formulation is consumed as an oxidant to promote anaerobic microbial respiration.

H13. The method of any of paragraphs H1-H12, wherein the method further includes creating an environment in which the contaminant is degraded during anaerobic microbial respiration.

H14. The method of paragraph H13, wherein the method further includes creating an environment in which the contaminant is oxidized during anaerobic microbial respiration.

H15. The method of any of paragraphs H1-H14, wherein the contaminant includes at least one of a hydrocarbon, a petroleum hydrocarbon, a metal, a partially halogenated solvent, and vinyl chloride.

H16. The method of any of paragraphs H1-H15, wherein the native microbe population includes at least one of bacteria, fungi, denitrifiers, sulfate reducers, anaerobic species, facultative anaerobic species, and facultative aerobic species.

H17. The method of any of paragraphs H1-H16, wherein the method further includes selecting a composition of the bioremediation formulation based on at least one of a characteristic of the contaminated region, a characteristic of the contaminant, and a characteristic of the native microbe population.

H18. The method of any of paragraphs H1-H17, the method further including identifying the contaminated region.

It is believed that the disclosure set forth above encompasses multiple distinct inventions with independent utility. While each of these inventions has been disclosed in its preferred form, the specific embodiments thereof as disclosed and illustrated herein are not to be considered in a limiting sense as numerous variations are possible. The subject matter of the inventions includes all novel and non-obvious combinations and subcombinations of the various elements, features, functions and/or properties disclosed herein. Similarly, where the claims recite “a” or “a first” element or the equivalent thereof, such claims should be understood to include incorporation of one or more such elements, neither requiring nor excluding two or more such elements.

It is believed that the following claims particularly point out certain combinations and subcombinations that are directed to one of the disclosed inventions and are novel and non-obvious. Inventions embodied in other combinations and subcombinations of features, functions, elements and/or properties may be claimed through amendment of the present claims or presentation of new claims in this or a related application. Such amended or new claims, whether they are directed to a different invention or directed to the same invention, whether different, broader, narrower, or equal in scope to the original claims, are also regarded as included within the subject matter of the inventions of the present disclosure.

1. A bioremediation formulation configured to provide at least a portion of the oxidants and nutrients that are consumed by a native microbe population during anaerobic respiration and to promote anaerobic oxidative bioremediation of a contaminant, the bioremediation formulation comprising:
a sulfate salt, wherein the sulfate salt comprises 20-60 wt % of the bioremediation formulation;
a nitrate salt, wherein the nitrate salt comprises 10-40 wt % of the bioremediation formulation;
a phosphate salt, wherein the phosphate salt comprises 10-30 wt % of the bioremediation formulation;
a complex sugar, wherein the complex sugar comprises 5-15 wt % of the bioremediation formulation; and
brewer's yeast, wherein the brewer's yeast comprises 5-15 wt % of the bioremediation formulation.

2. A bioremediation formulation configured to provide at least a portion of the oxidants and nutrients that are consumed by a native microbe population during anaerobic respiration and to promote anaerobic oxidative bioremediation of a contaminant contained within a treatment zone that is associated with a contaminated region, the bioremediation formulation comprising:
a high-mobility oxidant, wherein the high-mobility oxidant has a high-mobility oxidant diffusion constant when present within the treatment zone;
a low-mobility oxidant, wherein the low-mobility oxidant has a low-mobility oxidant diffusion constant when present within the treatment zone, and further wherein the low-mobility oxidant diffusion constant is less than the high-mobility oxidant diffusion constant; and
a nutrient material, wherein the nutrient material includes brewer's yeast.

3. The bioremediation formulation of claim 2, wherein the bioremediation formulation further includes a mid-mobility oxidant, wherein the mid-mobility oxidant has a mid-mobility oxidant diffusion constant when present within the treatment zone, wherein the mid-mobility oxidant diffusion constant is less than the high-mobility oxidant diffusion constant, and further wherein the mid-mobility oxidant diffusion constant is greater than the low-mobility oxidant diffusion constant.

4. The bioremediation formulation of claim 2, wherein the brewer's yeast comprises 5-15 wt % of the bioremediation formulation.

5. The bioremediation formulation of claim 2, wherein the nutrient material includes at least a first complex sugar.

6. The bioremediation formulation of claim 5, wherein the at least a first complex sugar comprises 5-15 wt % of the bioremediation formulation.

7. The bioremediation formulation of claim 2, wherein the high-mobility oxidant comprises 10-40 wt % of the bioremediation formulation.

8. The bioremediation formulation of claim 2, wherein the high-mobility oxidant includes a nitrate salt.

9. The bioremediation formulation of claim 2, wherein the low-mobility oxidant comprises 20-60 wt % of the bioremediation formulation.

10. The bioremediation formulation of claim 2, wherein the low-mobility oxidant includes a sulfate salt.

11. The bioremediation formulation of claim 2, wherein the bioremediation formulation further includes at least a first phosphate salt.

12. The bioremediation formulation of claim 11, wherein the at least a first phosphate salt comprises 10-30 wt % of the bioremediation formulation.

13. The bioremediation formulation of claim 11, wherein a nitrogen to phosphorous ratio in the bioremediation formulation is between 3:1 and 6:1.

14. An aqueous bioremediation solution, the solution comprising:
water; and
the bioremediation formulation of claim 2.

15. The aqueous bioremediation solution of claim 14, wherein the bioremediation formulation comprises 10-25 wt % of the aqueous bioremediation solution.

16. An aquifer comprising:
water;
a contaminant; and
the bioremediation formulation of claim 2.

17. The aquifer of claim 16, wherein the contaminant includes a hydrocarbon, and further wherein the aquifer includes 0.5-5 kilograms of the bioremediation formulation for each kilogram of the contaminant present within the aquifer.

18. A method of supplying an oxidant and nutrients to a native microbe population to promote consumption of a contaminant by anaerobic oxidative bioremediation, wherein the contaminant is contained within a treatment zone that is associated with a contaminated region, the method comprising:
estimating a mass of contaminant present within the treatment zone; and
supplying a mass of the bioremediation formulation of claim 1 to the treatment zone, wherein the supplying includes supplying 0.5-5 kilograms of the bioremediation formulation for each kilogram of the contaminant present within the treatment zone.

19. A method of supplying an oxidant and nutrients to a native microbe population to promote consumption of a contaminant by anaerobic oxidative bioremediation, wherein the contaminant is contained within a treatment zone that is associated with a contaminated region, the method comprising:
estimating a mass of contaminant present within the treatment zone; and
supplying a mass of bioremediation formulation to the treatment zone, wherein the supplying is based at least in part on the estimating, wherein the bioremediation formulation includes a high-mobility oxidant, a low-mobility oxidant, and a nutrient material, wherein the nutrient material includes brewer's yeast, and further wherein the supplying supports the anaerobic oxidative bioremediation of the contaminant.

20. The method of claim 19, wherein the supplying includes supplying between 0.5 and 5 kilograms of bioremediation formulation per kilogram of contaminant present within the treatment zone.

21. The method of claim 19, wherein the method further includes mixing the bioremediation formulation with water to form an aqueous bioremediation solution, wherein the bioremediation formulation comprises 10-25 wt % of the aqueous bioremediation solution, and further wherein the supplying includes supplying the aqueous bioremediation solution to the treatment zone.

22. The method of claim 19, wherein the method further includes providing a chemical oxidant to the treatment zone and oxidizing the contaminant with the chemical oxidant.

23. The method of claim 19, wherein the method further includes selecting a composition of the bioremediation formulation based on at least one of a characteristic of the contaminated region, a characteristic of the contaminant, and a characteristic of the native microbe population.

24. The method of claim 19, the method further including identifying the contaminated region.
